(19) United States
(12) Patent Application Publication
(10) Pub. No.: US 2009/0214424 A1
(43) Pub. Date: Aug. 27, 2009

(54) Human glycoprotein hormone superagonists and uses thereof

(76) Inventors: Mariusz W. Szkudlinski, Rockville, MD (US); Bruce D. Weintraub, Rockville, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 10/594,843

(22) PCT Filed: Mar. 18, 2005
(86) PCT No.: PCT/US2005/008957

§ 371 Date: Sep. 28, 2006

Related U.S. Application Data
(60) Provisional application No. 60/557,704, filed on Mar. 31, 2004.

(51) Int. Cl. A61K 51/08; A61K 49/00; A61K 49/14; A61K 49/04; A61K 38/14; A61K 39/395; A61K 35/76; A61K 38/21; A61K 38/20; G01N 33/53

(52) U.S. Cl. 424/1.69; 424/9.1; 424/9.6; 424/9.34; 424/9.43; 514/8; 424/178.1; 424/93.6; 424/85.7; 424/85.6; 424/85.5; 424/85.2; 436/501; 435/7.1; 435/7.2

(57) ABSTRACT

The present invention provides improved methods of imaging, targeted therapy and detection and diagnostics using modified glycoprotein hormones having increased activity over wild-type hormones.

### 1. FIELD OF THE INVENTION

The present invention provides methods of imaging cells comprising a glycoprotein hormone receptor and methods of assaying for an analyte that interferes with the binding of a modified glycoprotein hormone to a glycoprotein hormone receptor. The present invention also provides methods of targeted delivery of an agent coupled to a modified glycoprotein hormone to a subject in need thereof.

### 2. BACKGROUND

Thyroid-stimulating hormone (thyrotropin, TSH), chorionic gonadotropin, (CG), luteinizing hormone (lutropin, LH), and follicle-stimulating hormone (follitropin, FSH) comprise the family of glycoprotein hormones. Each hormone is a heterodimer of two non-covalently linked subunits: α and β. Within the same species, the amino acid sequence of the α-subunit is identical in all the hormones, whereas the sequence of the β-subunit is hormone specific. (Pierce and Parsons, Ann. Rev. Biochem. 1981, 50: 465-495).

These hormones were originally purified from the anterior pituitary (TSH, LH, and FSH) and placenta (CG) and shown to activate specific G protein-coupled receptors in the thyroid (TSH receptor) and gonads (LH and FSH receptors), respectively. (Greep, et al. Anat. Rec. 1936, 65: 261-71, Simpson, et al. Anat. Rec. 1950, 106: 247-48, Pierce, et al. Recent Prog. Horm. Res. 1971, 27: 165-212 and Shupnik, et al. Endocr. Rev. 1989, 10: 459-75). These three pituitary-derived glycoprotein hormones form the basis of the classic pituitary-peripheral target feedback systems and are essential for the development and differentiation of thyroid and gonadal tissues. (Weetman, N. Engl. J. Med. 2000, 343: 1236-48 and Paschke and Ludgate, N. Engl. J. Med. 1997, 337: 1675-81).

In some carcinomas, autoimmune disorders or fertility disorders, glycoprotein receptors are present in higher than normal quantities possibly due to gene overexpression. See, for example, Meier, et al., J. Clin. Endocrinol. Metabol. 1994, 78:188-196 and Yamamoto, et al., Hepatology 2003, 37: 528-33. Currently, detecting or diagnosing such disorders often involves imaging or in vitro assaying that is less specific or less sensitive than desired. More sensitive and specific methods of imaging, detecting, diagnosing and assaying disorders associated with production or expression of glycoprotein hormone receptors are needed. See, for example, Castellani, et al., Tunori 2003, 89(5):560-2 and Mendez, et al., Cancer 2004, 100(4):710-4 and Kahn, et al., Chest 2004, 125(2):494-501.

In addition, treatment of disorders involving autoantibody production to glycoprotein-receptors and disorders associated with glycoprotein hormones do not target the desired tissue. Rather, these treatments often cause unwanted side effects. For example treatment of thyroid carcinoma with iodine 131 is associated with hematopoietic system depression, thyroid crisis, chest pain, tachycardia, rash, hives, dysphagia and alopecia. See, Drug Facts and Comparisons, Updated Monthly, (March, 2004) Wolters Kluwer Company, St. Louis, Mo. More effective ways to treat these disorders and provided targeted delivery of therapeutic agents are needed.

### 3. SUMMARY OF THE INVENTION

The present invention provides methods of imaging and detecting cells comprising a glycoprotein hormone receptor and methods of assaying for an analyte that interferes with the binding of a modified glycoprotein hormone to a glycoprotein receptor. The present invention also provides methods of targeted delivery of an agent coupled to a modified glycoprotein hormone to a subject in need thereof.

The present invention provides methods of imaging cells comprising a glycoprotein hormone receptor, said method comprising administering to a subject a modified glycoprotein hormone, said modified glycoprotein hormone having at least one mutation that increases the hormone activity relative to the wild type glycoprotein hormone and detecting said modified glycoprotein hormone.

In certain embodiments, the methods provide for imaging cells comprising a glycoprotein hormone receptor wherein the cells are cancerous cells or cells indicative of an autoimmune disorder. In certain embodiments, the methods of imaging provide that detecting increased levels of said modified glycoprotein hormone in said subject indicates the presence of cancerous cells or an autoimmune disorder. In certain embodiments of the invention, the methods of imaging a cell comprising a glycoprotein hormone receptor provide that the modified glycoprotein hormone is labeled. In certain embodiments, the methods provide that detecting an amount of a labeled modified glycoprotein hormone in a subject indicates the presence of cancerous cells or an autoimmune disorder.

The present invention also provides methods of delivering an agent to a cell expressing a glycoprotein receptor to a subject in need thereof, said method comprising administering to said subject an agent coupled to a modified glycoprotein hormone having at least one mutation that increases the hormone activity relative to the wild type glycoprotein hormone. This method is also referred to as a method of targeted delivery of an agent.

The present invention also provides methods for the detection of an analyte that interferes with the binding of a modified glycoprotein hormone to a glycoprotein receptor in a biological sample, said method comprising (i) contacting the sample with a modified glycoprotein hormone, said modified glycoprotein hormone having at least one mutation that increases the hormone activity relative to the wild type glycoprotein hormone and (ii) detecting a signal wherein the presence or amount of the signal detected indicates the presence or absence of an analyte that interferes with the binding of a modified glycoprotein hormone to a glycoprotein receptor. In one embodiment, the methods provide that the signal to be detected is the presence or amount of the modified glycoprotein hormone bound with the glycoprotein receptor in the biological sample. In certain embodiments, the methods provide for the detection of a secondary signal, such as, for example, the presence or amount of cAMP or steroids (e.g., progesterone).

In certain embodiments, the methods provide for the detection of an analyte wherein the analyte is an antibody to a glycoprotein receptor or fragments thereof. In certain embodiments the methods provide, inter alia, for the detection of an antibody to a glycoprotein hormone receptor extracellular domain or fragment thereof. In certain embodiments, the methods provide for the detection of an analyte wherein the analyte is wild type glycoprotein hormone. In certain embodiments, the methods provide that the glycoprotein receptor can be the receptor for TSH, FSH, LH, CG or combinations thereof.

The methods of the invention comprise the use of modified glycoprotein hormones. In certain embodiments, the methods provide that the modified glycoprotein hormone can be a modified thyroid stimulating hormone (TSH), a modified follicle-stimulating hormone (FSH), a modified leutenizing hormone (LH) or a modified chorionic gonadotropin (CG) as described herein.

### 4. BRIEF DESCRIPTION OF THE FIGURE

FIG. 1 provides a schematic depicting the TSH receptor (TSHR)-mediated delivery of various therapeutic agents to a thyroid cancer cell.

### 5. DETAILED DESCRIPTION OF THE INVENTION

The modified glycoprotein hormones useful in the methods of the invention have increased activity over wild-type glycoprotein hormones. The relative activity (e.g., potency) of the modified glycoprotein hormones as compared with the wild-type glycoprotein hormone is at least about 3 fold to at least about 6 fold higher. In addition the modified glycoprotein hormones have a high affinity for glycoprotein receptors. These attributes of the modified glycoprotein hormones care be exploited in the present invention to provide improved methods of imaging, detecting and assaying cells involved in glycoprotein hormone related disorders as well as methods of delivering agents to cells involved in glycoprotein hormone related disorders.

The present invention provides methods of imaging and detecting cells comprising a glycoprotein hormone receptor and methods of assaying for an analyte that interferes with the binding of a modified glycoprotein hormone to a glycoprotein receptor. The present invention also provides methods of targeted delivery of a therapeutic agent coupled to a modified glycoprotein hormone to a subject in need thereof.

A. Methods of Imaging

In one embodiment, the invention provides methods of imaging cells comprising a glycoprotein hormone receptor, said method comprising administering to a subject a modified glycoprotein hormone, said modified glycoprotein hormone having at least one mutation that increases the hormone activity relative to the wild type glycoprotein hormone and detecting said modified glycoprotein hormone. The method of imaging and detecting the hormone can be any method known to those of skill in the art. Commonly used imaging methods include, for example, magnetic resonance imaging (MRI), X-ray, computed tomography (CT), positron emission tomography (PET), mammography and ultrasound.

Methods of imaging subjects using basic radiologic techniques have been described, for example, “Textbook of Radiology and Imaging,” Sutton and Livingstone, 7th Edition, (2 Volume set), Churchill Livingstone (Elsevier Sciences), London, 2002, “A Concise Textbook of Radiology,” Armstrong and Wastie (eds.) Arnold Publishing (The Thomson Corporation), Scarborough, Ontario, Canada, 2001, “Walter & Miller's Textbook of Radiotherapy,” Bomford and Knuckler, 6th Edition, Churchill Livingstone (Elsevier Sciences), London, 2001, incorporated herein by reference in their entireties. See also, Bottomley, Comput. Radiol. 1984, 8(2): 57-77, Dixon, Radiology 1984, 153(1): 189-94, Daley and Cohen, Cancer Res. 1989, 49(4):770-9, Ellis, et al., Clin. Radiol. 2001, 56(9):691-9, Paushter, et al., Med. Clin. North Am. 1984, 68(6):1393-421, Blecher, Aust. Fam. Physician 1983 12(6):449-50, 452, Bragg, Cancer 1977, 40(1 Suppl):500-8, Moseley, Br. Med. J. (Clin. Res. Ed.) 1982, 284(6323):1141-4, Lentle and Aldrich, Lancet 1997, 350(9073):280-5, Weber, et al., Strahlenther Onkol. 1999, 75(8):356-73, Hanbidge, Can. J. Gastroenterol. 2002, 16(2):101-5, Miles, Eur. Radiol. 2003, Suppl 5:M134-8, Prigent-Le Jeune, et al., Eur. J. Nucl. Med. Mol. Imaging. 2004, Feb. 19 [Epub ahead of print], DeSimone, et al., Gynecol. Oncol. 2003, 89(3):543-8 and Goldenberg, et al., J. Clin. Oncol. 1987, 5(11): 1827-35, incorporated herein by reference in their entireties.

Any suitable means of imaging or detecting can be employed, depending, inter alia, on the nature of the subject's disorder or suspected disorder, the tissue to be imaged and whether functional (physiologic) or structural (anatomic) images are desired. In some embodiments, among others, the methods of imaging provide that detecting an amount of a labeled modified glycoprotein hormone in a subject or detecting increased levels of a modified glycoprotein hormone in a subject indicates the presence of cancerous cells or an autoimmune disorders elected from the group consisting of thyroid cancer, Graves' disease, Hashimoto's disorder, ovarian cancer, uterine cancer, cervical cancer, endometrial cancer, lung cancer, teratomas, breast cancer, testicular cancer or pituitary tumor.

Imaging methods can be broadly categorized as those that provide information regarding the structure or anatomy of a subject or those that provide function or physiology of a subject. Structural imaging provides the shape of a bone or tissue component to determine, for example, if there are abnormal formations or destruction of certain elements. Tumors or the presence of cancerous cells can app ear as structural changes. A newer type of structural imaging provides the chemical composition of different parts of a tissue in order to determine if there is ongoing injury or abnormal biochemical processes (e.g., presence or growth of cancerous cells). See, for example, Bonilha, et al., Med. Sci. Monit. 2004, 10(3):RA40-6, epub 2004 Mar. 1, Ballmaier, et al., Psychiatry Res. 2004, 15; 130(1):43-55, Ballmaier, et al., Biol. Psychiatry, 2004, 55(4):382-9, Cha, Magn. Reson. Imaging Clin. N. Am. 2003, 11(3):403-13 and Kopelman, et al., Hippocampus, 2003; 13(8):879-91, incorporated herein by reference in their entireties.

Functional imaging is a relatively new technique which seeks to ascertain whether particular tissues or organs are performing particular functional tasks. This technique can capitalize on a number of physiologic processes, including, for example, blood flow and activity-associated with changes in blood flow (i.e., neoplastic presence or growth) and monitoring responses to chemotherapy. See, for example, Takeuchi, et al., J. Med. Invest. 2004, 51(1-2):59-62, Otsuka, et al., J. Med. Invest. 2004, 51(1-2): 14-9, Martincich, et al., Breast Cancer Res. Treat. 2004, 83(1):67-76, Cohen and Goadsby, Curr. Neurol. Neurosci. Rep. 2004, 4(2): 105-10 and Lewis, et al., Eur. J. Neurosci. 2004, 19(3):755-60, incorporated herein by reference in their entireties.

Without being bound by any theory, it is expected that a specific sub-group of subjects in particular will benefit from the methods of the invention. These subjects are those with decreased glycoprotein hormone receptor binding due to mutations in the receptor that decrease glycoprotein hormone binding and/or glycoprotein hormone receptor expression. High affinity glycoprotein analogs, such as the modified glycoproteins described herein, are expected to overcome, at least in part, limitations of imaging and targeted delivery of an agent in such a sub-group of subjects.

In certain embodiments, the subject is a mammal. In preferred embodiments, the subject is human.

In general, radiological methods such as, for example, magnetic resonance imaging (MRI), X-ray, computed tomography (CT), mammography and ultrasound provide structural or anatomic information regarding a subject. Radiological methods such as, for example, nuclear medicine, radionuclide imaging and positron emission tomography (PET) provide functional or physiologic information regarding a subject. Both structural and functional imaging are within the scope of the present invention.

In one embodiment of the invention, the imaging methods provide that the modified glycoprotein hormone is labeled (i.e., a contrast agent is used). Any label or contrast agent can be used. See, Minato, et al. J. Comput. Assist. Tomogr. 2004, 28(1):46-51, Antoch, et al., JAMA 2003, 290(24):3199-206, Brinker, Rev. Cardiovasc. Med. 2003; 4 Suppl 5:S19-27, el-Diasty, et al., J. Urol. 2004, 171(1):31-4, Williams, et al., Int. J. Oral Maxillofac. Surg. 2003, 32(6):651-2, Follen, et al., Cancer 2003, 98(9 Suppl):2028-38, Behrenbruch, et al., Med. Image Anal. 2003, 7(3):311-40, Knopp, et al., Mol. Cancer. Ther. 2003, 2(4):419-26, incorporated herein by reference in their entireties. The label can be any label known to those of skill in the art. In one embodiment, the label can be a radiopaque label, radioactive label, fluorescence label or paramagnetic label. Radiopaque labels are those which are not transparent to X-rays or other radiation (e.g., MRI) and are usually grouped according to osmolality (high or low), structure (monomeric or dimeric ring structure), and ion tendency (nonionic or ionic).

X-ray radiography contrast agents are generally dyes that absorb X-rays, making the organs containing them visible in contrast to the surrounding tissue. High osmolality contrast media have an osmolality in solution between 1200 and 2400 mOsm/kg water and are ionic monomers. Low osmolality contrast media are classified as ionic dimers (i.e., ioxaglate), nonionic monomers or nonionic dimers. Because of lower toxicities nonionic monomers are becoming the more preferred contrast media. The nonionic dimers are still mostly in the developmental stages but they are of limited clinical use because of their viscosity approaching that of plasma. The osmolality of low osmolality contrast media is about 290 to 860 mOsm/kg water. The most important characteristic of contrast media is the iodine content. The relatively high atomic weight of iodine contributes sufficient radiodensity for radiographic contrast with surrounding tissues. See, Drug Facts aid Comparisons, Updated Monthly, (March, 2004) Wolters Kluwer Company, St. Louis, Mo., incorporated herein by reference in its entirety.

Osmolality and Viscosity of Radiopaque Agents

| | | |
|---|---|---|
| | | Viscosity |
| | Osmolality | (cps at |
| Radiopaque Agent | (mOsm/kg H₂O) | 37° C.) |

Ionic Agents

| | | |
|---|---|---|
| Diatrizoate meglumine 30% | 633 | 1.42 |
| Diatrizoate meglumine 60% | 1415 | 4.12 |
| Diatrizoate meglumine 66% and | 2016 | 9.0 |
| diatrizoate sodium | | |
| 10% (Hypague-76 ®) | | |
| Diatrizoate meglumine 66% and | 1551 | 10.5 |
| diatrizoate sodium 10% (MD-76R ®) | | |
| Diatrizoate meglumine 66% and | 1870 | 9.1 |
| diatrizoate sodium 10% (RenoCal-76 ®) | | |
| Diatrizoate sodium 50% | 1515 | 2.34 |
| Iothalamate meglumine 30% | 600 | 1.5 |
| Iothalamate meglumine 43% | 1000 | 2.0 |
| Iothalamate meglumine 60% | 1400 | 4.0 |
| Iothalamate meglumine 39.3% and | 600 | 7.5 |
| ioxaglate sodium 19.6% | | |

Nonionic agents

| | | |
|---|---|---|
| Gadodiamide | 789 | 1.4 |
| Gadoteridol | 630 | 1.3 |
| Gadoversetamide | 1110 | 2.0 |
| Iodixanol 270 | 290 | 6.3 |
| Iodixanol 320 | 290 | 11.8 |
| Iohexol 140 | 322 | 1.5 |
| Iohexol 180 | 408 | 2.0 |
| Iohexol 240 | 520 | 3.4 |
| Iohexol 300 | 672 | 6.3 |
| Iohexol 350 | 844 | 10.4 |
| Iopamidol 41% | 413 | 2.0 |
| Iopamidol 51% | 524 | 3.0 |
| Iopamidol 61% | 616 | 4.7 |
| Iopamidol 76% | 796 | 9.4 |
| Iopromide 150 | 328 | 1.5 |
| Iopromide 240 | 483 | 2.8 |
| Iopromide 300 | 607 | 4.9 |
| Iopromide 370 | 774 | 10.0 |
| Ioversol 34% | 355 | 1.9 |
| Ioversal 51% | 502 | 3.0 |
| Ioversol 64% | 651 | 5.5 |
| Ioversol 68% | 702 | 5.8 |
| Ioversol 74% | 792 | 9.0 |

Paramagnetic agents

| | | |
|---|---|---|
| Ferumoxides | 340 | — |
| Gadopentetate dimeglumine | 1960 | 2.9 |
| Mangofodipir trisodium | 298 | 0.8 |

adapted from Drug Facts and Comparisons, Updated Monthly, (March, 2004) Wolters Kluwer Company, St. Louis, Missouri, p. 2003.

In one embodiment, the radiopaque label is an ionic or nonionic agent. A number of ionic and nonionic agents are available and can be used in the methods of the invention. For example, an ionic agent can be diaztrizoate meglumine 30%, diaztrizoate meglumine 60%, diaztrizoate meglumine 66% and diaztrizoate sodium 10%, diaztrizoate sodium 50%, iothalamate meglumine 30%, iothalamate meglumine 43%, iothalamate meglumine 60%, ioxaglate meglumine 39.3%, iothalamate sodium 19.6% or combinations thereof. In one embodiment, the nonionic agents can be, for example, gadodiamide, gadoteridol, gadoversetamide, iodixanol 270, iodixanol 320, iohexyl 140, iohexyl 180, iohexyl 240, iohexyl 300, iohexyl 350, iopamidol 41%, iopamidol 51%, iopamidol 61%, iopamidol 76%, iopromide 150, iopromide 240, iopromide 300, iopromide 370, ioversol 34%, ioversol 51%, ioversol 64%, ioversol 68%, ioversol 74% or combinations thereof.

Contrast agents for magnetic resonance imaging are paramagnetic agents that influence the longitudinal or spin-lattice (T₁) time or the transverse or spin-spin relaxation time (T₂). Paramagnetic contrast agents generally act by decreasing the T₁ or T₂ values in tissues that retain the contrast agents, enhancing the signal intensity. See, Drug Facts and Comparisons, Updated Monthly, (March, 2004) Wolters Kluwer Company, St. Louis, Mo. and Physicians' Desk Reference Medical Economics Data, Montvale, N.J. 1993, incorporated herein by reference in their entireties. Any agent that affects T₁ or T₂ times can be used in the methods of the invention. In one embodiment, the paramagnetic labels used in the methods of the invention can be, for example, ferumoxides (FERIDEX I.V.® Berlex), gadopentetate dimeglumine (MAGNEVIST,® Berlex), mangafodipir tridosium (TESLASCAN®, Nycomed) or combinations thereof.

Nuclear medicine involves the use of radioisotopes, either alone or bound to a biological molecule that has some known biologic function (radiopharmaceuticals), often to study physiologic changes in the body. As used herein the terms radioisotope, radiopharmaceutical and radionuclide will be used interchangeably. The radiopharmaceuticals are administered to the subject usually by venous injection (e.g., intravenously). Once injected, the radiopharmaceuticals participate in the physiologic processes taking place in various organs and tissues. The imaging systems then detect the radioactive emissions (usually beta (β) or gamma (γ) radiation) to create an image. Examples of clinically useful radioisotopes are iodine 131 (I¹³¹) and Technecium 99m (Tc^{99m}).

The radionuclides generally will be in the form of a stable complex, e.g., a chelate. The biodistribution of such diagnostic agents in vivo can be analyzed by appropriate standard external (i.e., non-invasive) means. In a preferred embodiment, the radioisotope labels are I¹³¹ or Tc^{99m}.

Radionuclides generally emit either beta (p) or gamma (γ) radiation. I¹³¹ emits about 90% β-radiation and about 10% γ-particles and has a physical half life of about 8 days. Tc^{99m} emits γ-radiation and has a half life of about 6 hours. Following administration of, for example, a Tc^{99m}-labeled antibody, the biodistribution of the radionuclide can be detected by scanning the patient with a gamma camera using known procedures. Accumulations of Tc^{99m} at the target site(s) is thus easily imaged. See, Toohey, Radiographics 2000; 20:533-546, Kostakoglu, et al., RadioGraphics 2003, 23:315-340, Saremi, et al., RadioGraphics 2002, 22:477-490, Intenzo, et al., RadioGraphics 2001, 21:957-964, Ranger, RadioGraphics 1999, 19:481-502, Simpkin, RadioGraphics 1999, 19:155-167, Janoki and Kerekes, Acta Physiol. Hung 1992, 79(2):183-96, Hoefnagel, Anticancer Drugs 1991, 2(2):107-32, Hoefnagel, Eur. J. Nucl. Med. 1991, 18(6):408-31, Gatley, et al., Acta Radiol. Suppl. 1990, 374:7-11, Ott, Br. J Radiol. 1989, 62(737):421-32, Andersen, Cerebrovasc. Brain Metab. Rev. 1989, 1(4):288-318 and Miraldi, Int. J. Radiat. Oncol. Biol. Phys. 1986, 12(7): 1033-9, incorporated herein by reference in their entireties.

In addition to I¹³¹ or Tc^{99m}; any radioisotope known to those of skill in the art can be employed in the methods of the invention. Other radionuclides and chelates can include, for example, Co⁵⁷, Co⁵⁸, Cr⁵¹, F¹⁸ FDG, Ga⁶⁷, In¹¹¹ chloride, In¹¹¹ pentetate (DTPA), In¹¹¹ oxyquinoline (oxine), In¹¹¹ Capromab pendetide, In¹¹¹ Imciroma pentetate, In¹¹¹, pentetreotide, In¹¹¹ satumomab pendetide, I¹²³, I¹²⁵ iothalamate, I¹²⁵ human serum albumin (RISA), I¹³¹ iodohippurate, I¹³¹ iodomethylnorcholesterol (NP-59), I¹³¹ metaiodobenzylguanidine (MIBG), Kr^{81m} gas, P³² chromic phosphate, P³² sodium phosphate, Ru⁸², Sm¹⁵³ lexidronam (Sm-153 EDTMP), Sr⁸⁹, Tl²⁰¹ and Xe¹³³.

Any chelate of a radionuclide can be used in the methods of the invention. For example, although Tc^{99m} pertechnetate is one of the most common forms of Tc^{99m} used clinically, other forms of Tc^{99m} are available and within the scope of the invention, such as, Tc^{99m} DMSA (dimercapto succinic acid), Tc^{99m} Apcitide, Tc^{99m} Arcitumomab, Tc^{99m} albumin colloid, Tc^{99m} bicisate (ECD), Tc^{99m} Depreotide, Tc^{99m} disofenin (DISIDA), Tc^{99m} exametazine (HMPAO), Tc^{99m} Gluceptate, Tc^{99m} Human Serum Albumin (HSA), Tc^{99m} Lidofenin (HIDA), Tc^{99m} Macroaggregated Albumin (MAA), Tc^{99m} Mebrofenin, Tc^{99m} Medronate (MDP), Tc^{99m} Mertiatide, Tc^{99m} Nofetumomab Merpentan, NR-LU-10, Tc^{99m} Oxidronate (HDP), Tc^{99m} Pentetate (DTPA), Tc^{99m} Pyrophosphate (PYP), Tc^{99m} Red Blood Cells (RBCs), Tc^{99m} Sestamibi, Tc^{99m} Succimer (DMSA), Tc^{99m} Sulfur Colloid (SC), Tc^{99m} Teboroxime or Tc^{99m} Tetrofosmin.

Other available imaging, diagnostic or contrast agents, preferably those commercially available can be used in the methods of the invention. Commercially available agents used to diagnose, monitor and evaluate thyroid and gonadotropin disorders are preferred. Such agents include, for example, protirelin (THYPINONE®, Abbott, and others), thyrotropin alpha (THYROGEN®, Genzyme) or gonadorelin (FACTREL®, American Home Products) or combinations thereof.

In certain embodiments, the methods provide for detecting an unlabeled modified glycoprotein hormone. The detection of the unlabeled modified glycoprotein hormone can be made by one of skill in the art. For imaging methods such as CT and MRI, the use of a contrast agent or label is optional. When a noncontrast CT or MRI is employed, differences between tissues (tissue contrast) can be observed based on tissue density. With noncontrast CT, tissue contrast is provided by variations in the density of the tissue being examined. Denser tissues (e.g., bone, foreign bodies or tumors) appear white on CT and less dense tissues (e.g., air or water) appear black. In noncontrast MRI, the T₁ and T₂ relaxation times of various tissues determine tissue contrast (i.e., the lightness or darkness of the image). With ultrasound, highly dense tissues, such as bone or kidney stones, reflect echoes and, therefore, appear white on an ultrasound image. Air, such as in the bowel, also reflects echoes, so the edge of the bowel appears white on an ultrasound image. Thus, substances with widely differing densities (e.g., air, bone) may appear bright white on an ultrasound image. The ability to detect an unlabeled modified glycoprotein hormone using noncontrast imaging methods is within the capabilities of one of skill in the art, especially in light of the detailed description provided herein.

B. Methods of Delivering an Agent

The present invention provides a method of delivering an agent to a cell expressing a glycoprotein receptor to a subject in need thereof, said method comprising administering to said subject an agent coupled to a modified glycoprotein hormone having at least one mutation that increases the hormone activity relative to the wild type glycoprotein hormone. The method of delivering an agent to a cell (i.e., targeted delivery) can employ any suitable agent, depending on the nature of the subject's illness or suspected illness. The agent can be a cytoprotective compound, antibody, drug, sensitizer, biological response modifier, radionuclide, toxin, viruses or combination thereof.

In certain embodiments, the methods of targeted delivery are for the treatment of a subject with a disorder or suspected disorder associated with abnormal glycoprotein receptor expression. In certain embodiments, the methods of targeted delivery are for the diagnosis or detection of a disorder associated with abnormal glycoprotein receptor expression. In certain embodiments, the methods of targeted delivery can be used in conjunction with other therapies, diagnostic procedures or clinical modalities, including radiation and/or surgery (e.g., transsphenoidal surgery of the pituitary, reduction mammaplasty, mastectomy, hysterectomy, and the like).

In certain embodiments, the methods provide for the restoration of cancer cell differentiation. Without being bound to any theory, it is hypothesized that delivery of genetic material can be facilitated by the high affinity interaction between the modified glycoprotein hormones described herein and the glycoprotein hormone receptors. In certain embodiments, genetic material can be coupled to a modified glycoprotein hormone for targeted delivery to a cancerous cell. The uptake of this genetic material can increase the number of receptors and restore cell differentiation. It is also hypothesized that delivery of a modified glycoprotein hormone to a cancerous cell, for example, delivery of modified TSH to a thyroid cancer cell, will increase the number of TSH receptors and stimulate or restore cell differentiation.

Without being bound by any theory, it is expected that a specific sub-group of subjects in particular will benefit from the targeted delivery methods of the invention. These subjects are those with decreased glycoprotein hormone receptor binding due to mutations in the receptor that decrease glycoprotein hormone binding and/or glycoprotein hormone receptor expression. High affinity glycoprotein analogs, such as the modified glycoproteins described herein, are expected to overcome, at least in part, limitations of providing agents to such a sub-group of subjects.

In certain embodiments, the subject is a mammal. In preferred embodiments, the subject is human.

In one embodiment, the methods provide for targeted delivery of an agent, wherein the agent is a cytoprotective compound. Cytoprotective compounds are those compounds which act to protect or decrease the incidence or severity of injury to a cell. Commercially available cytoprotective compounds include mesna (MESNEX®, Bristol-Myers Squibb), amifostine (ETHYOL®, Alza), dexrazoxane (ZINECARD®, Pharmacia & Upjohn) and leucovorin (multiple manufacturers). Mesna is a compound used to decrease the incidence of hemorrhagic cystitis in subjects receiving high dose cyclophosphamide. The cytoprotective compound amifostine, is used for the reduction of cumulative renal toxicity associated with repeated administration of cisplatin and for the reduction of the incidence of moderate-to-severe xerostomia in subjects undergoing postoperative radiation treatment. Amifostine is also used to protect lung fibroblasts from the damaging effects of paclitaxel. Dexrazoxane is used for the reduction of the incidence and severity of cardiomyopathy associated with doxorubicin administration in subjects. In particular, women treated with doxorubicin, for the treatment of, for example, metastatic breast cancer, that have received a cumulative doxorubicin dose of 300 mg/m² are preferred subjects for the administration of dexrazoxane. Leucovorin rescue is given after administration of methotrexate therapy in the treatment of osteosarcoma and after 5-fluorouracil administration in subjects with metastatic colorectal cancer. In preferred embodiments of the invention, the methods can employ the cytoprotective compounds, mesna, amifostine, dexrazoxane, leucovorin or combinations thereof.

The present invention provides, inter alia, methods of targeted delivery of an agent to a cell expressing a glycoprotein receptor. In one embodiment, the agent can be any drug used to treat various forms of cancer, such as, for example, natural or synthetic estrogens, estrogen receptor modulators, progestins, androgens, gonadotropin-releasing hormones, androgen inhibitors, bisphosphonates, glucocorticoids, thyroid hormones, antithyroid agents, iodine agents, bromocriptine, alkylating agents, antimetabolites, antimitotic agents, epipodophyllotoxins, antineoplastic antibiotics, antineoplastic hormones, platinum coordination complex agents, anthracenediones, substituted ureas, methylhydrazine derivatives, DNA topoisomerase inhibitors, retinoids, porfimer, mitotane or combinations thereof.

In one embodiment, the agent can be any drug used to treat cancers. In certain embodiments, the cancer can be thyroid carcinoma, pituitary adenomas (e.g., tumors), lung cancer, teratomas or cancers of the male or female reproductive systems (e.g., endometrial cancer, uterine cancer, cervical cancer, breast cancer, testicular cancer). In a preferred embodiment, the agent can be clomiphene, finasteride, propylthiouracil, methimazole, bleomycin, vincristine, vinblastine, cisplatin, mitomycin, ifosfamide, cyclophosphamide, doxorubicin, paclitaxel, fluorouracil, carboplatin, epirubicin, altretamine, vinorelbine, mitoxantrone, prednisone or combinations thereof.

Drugs known to enhance the cytotoxic effect of certain anti-cancer drugs and radiopharmaceuticals can also be used. Such drugs are commonly referred to as sensitizers. Examples of sensitizers which enhance the activity of various therapeutic drugs (e.g., anti-cancer drugs) are buthionine sulfoximine and calcium channel blockers such as verapamil, and diltiazem. See, U.S. Pat. No. 4,628,047 and Important Advances in Oncology 1986, DeVita, et al., Eds., J. B. Lippincott Co., Philadelphia, pages 146-157 (1986), incorporated herein by reference in their entireties. Other sensitizers known in the art are metronidazole, misonidazole, certain 2-sulfamyl-6-nitrobenzoic acid derivatives, 2,6-disubstituted derivatives of 3-nitropyrazine, and certain isoindoledione compounds. See, U.S. Pat. Nos. 4,647,588; 4,654,369; 4,609,659 and 4,494,547, incorporated herein by reference in their entireties.

In certain embodiments, the agent can be a biological response modifier. Any biological response modifier can be used in the scope of the invention. Examples of biological response modifiers useful in the methods of the invention include, but are not limited to interferon-α, interferon-β, interferon-γ, tumor necrosis factor, lymphotoxin, interleukin-1, interleukin-2, interleukin-3, interleukin-4, interleukin-5, interleukin-6, p53 or combinations thereof.

In certain embodiments, the agent can be a cell signal transduction pathway modifier. The glycoproteins activate specific G protein-coupled receptors in the thyroid (TSH receptor) and gonads (LH and FSH receptors), respectively. (Greep, et al. Anat. Rec. 1936, 65: 261-71, Simpson, et al. Anat. Rec. 1950, 106: 247-48, Pierce, et al. Recent Prog. Horm. Res. 1971, 27: 165-212 and Shupnik, et al. Endocr. Rev. 1989, 10: 459-75). In certain embodiments, the cell signal transduction pathway can be a G protein pathway. See, Penela, et al., Cell Signal 2003, 15(11):973-81. The cell signal transduction pathway can be any cell signal transduction pathway known to one of skill in the art. See, for example, Krymskaya, Cell Signal 2003, 15(8):729-39, Fung, et al., Cell Signal 2003, 15(6):625-36, Yamamoto, et al., Cell Signal 2003, 15(6):575-83, Marino, et al., Cell Signal 2003, 15(5):511-7, Rochette-Egly, Cell Signal 2003, 15(4):355-66, the contents of each of which are incorporated herein by reference in their entireties. In certain embodiments, the cell signal transduction pathway can be the cAMP/protein kinase A (PKA) pathway or the protein kinase C (PKC) pathway.

In certain embodiments, the agent can be forskolin or other modifiers of the cAMP/protein kinase A (PKA) pathway. See, Woo, et al., Neurosci. Lett. 2004, 19; 356(3):187-90 and Johnston, et al., J. Neurochem. 2004, 88(6):1497-508, for example. In certain embodiments, the cell signal transduction pathway can be staurosporine, phorbol esters or other modifiers of protein kinase C (PKC) activity. In certain embodiments, the agent can be a steroid or non-steroidal anti-inflammatory drug, such as indomethacin, or other modifier of prostaglandin/leukotriene synthesis. See, for example, Sasson, et al., Biochem. Biophys. Res. Commun. 2003, 28; 311(4): 1047-56, Paik, et al., Adv. Exp. Med. Biol. 2002, 507:503-8 and Pouplana, et al., J. Comput. Aided Mol. Des. 2002, 16(10):683-709.

In certain embodiments, the agent can be an antibody. The antibody can be a monoclonal or polyclonal antibody. In certain embodiments, the antibodies can be humanized antibodies.

In certain embodiments, the antibody can be a chimeric construct. The making and using of chimeric antibodies has been described, for example, in U.S. Pat. Nos. 6,693,176; 6,420,113; 6,329,508; 6,120,767; 5,807,548; 5,750,078 and 5,637,288, incorporated herein by reference in their entireties. The chimeric monoclonal antibodies useful in the methods of the invention can be produced by any method, including, by recombinant DNA techniques. See generally, Robinson et al., PCT Patent Publication PCT/US86/02269; Akira, et al., European Patent Application 184,187; or Taniguchi, M., European Patent Application 171,496, incorporated herein by reference in their entireties. In certain embodiments the antibody can be a functional fragment of an antibody, for example, Fab₁, Fab₂, etc.

Examples of toxins which can be employed in the methods of the invention are ricin, abrin, diphtheria toxin, Pseudomonas exotoxin A, ribosomal inactivating proteins, and mycotoxins; e.g., trichothecenes. Trichothecenes are a species of mycotoxins produced by soil fungi of the class fungi imperfecti or isolated from Baccharus megapotamica. (Bamburg, Proc. Molec. Subcell Bio. 1983, 8:41-110, Jarvis and Mazzola, Acc. Chem. Res. 1982, 15:338-395, incorporated herein by reference in their entireties.) Therapeutically effective modified toxins or fragments thereof, such as those produced through genetic engineering or protein engineering techniques, can be used.

The radionuclides useful in the methods of the present invention are described supra.

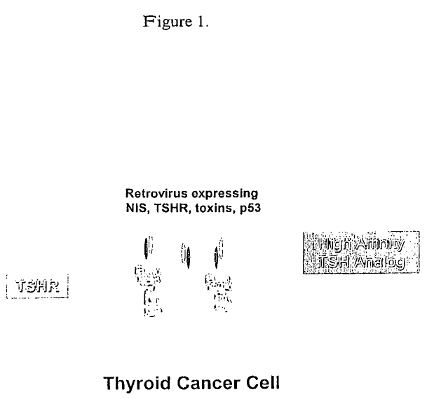

In certain embodiments, the methods provide, inter alia, for the targeted delivery of a virus coupled to a modified glycoprotein hormone. The retrovirus can be any virus suitable for the methods of the invention. In certain embodiments, the virus can be an adenovirus, retroviruses, lentiviruses, combinations or fragments thereof. See also, U.S. Pat. Nos. 6,399,385; 6,428,790 and 6,710,037, for example, describing uses of various viruses and fragments thereof. In certain embodiments, the virus can be a retrovirus that expresses an agent, for example, a glycoprotein hormone receptor or p53. In certain embodiments, the retrovirus is coupled to a modified glycoprotein hormone and coupled to an active agent, such as, sodium iodide symporter (NIS), toxins, or p53, as depicted in FIG. 1.

The methods of the invention provide, inter alia, for targeted delivery of an agent that is coupled to a modified glycoprotein hormone. Any means of coupling or linking an agent to a modified glycoprotein hormone can be employed. For example a number of different cleavable linkers have been described previously. See, U.S. Pat. Nos. 4,618,492; 4,542,225; and 4,625,014, incorporated herein by reference in their entireties. The mechanisms for release of an agent from these linker groups include by irradiation of a photolabile bond, and acid-catalyzed hydrolysis. U.S. Pat. No. 5,563,250, incorporated herein by reference in its entirety, discloses immunoconjugates comprising linkers of specified chemical structure, wherein the linkage is cleaved in vivo, releasing the compound (radiopharmaceutical, drug, toxin, etc.) in its native form. The linker is susceptible to cleavage at mildly acidic pH, and is believed to be cleaved during transport into the cytoplasm of a target cell, thereby releasing the biologically active compound inside a target cell. U.S. Pat. No. 4,671,958, incorporated herein by reference in its entirety, includes a description of immunoconjugates comprising linkers which are cleaved at the target site ill vivo by the proteolytic enzymes of the patient's complement system.

Other means of coupling or linking have been described. For example, tinker molecules are commercially available, such as those available from Pierce Chemical Company, Rockford, Ill. See Pierce 1986-87 General Catalog, pages 313-354, incorporated herein by reference in its entirety. Means for coupling to an antibody, (See, for example, U.S. Pat. Nos. 4,671,958 and 4,659,839, incorporated herein by reference in their entireties) and means of linking or coupling radionuclide metal chelates, toxins and drugs to proteins are known. See, for example, European Patent Application Publication No. 188,256; U.S. Pat. Nos. 4,671,958; 4,659,839, 4,414,148; 4,699,784; 4,680,338; 4,569,789; and 4,590,071; Borlinghaus et al. Canc. Res. 47:4071-4075, Aug. 1, 1987, Foran, Best Pract. Res. Clin. Haematol. 2002, 15(3): 449-65 and Fotiou, et al., Eur, J. Gynaecol. Oncol. 1988, 9(4): 304-7 incorporated herein by reference in their entireties. In view of the large number of methods that have been reported for coupling a variety of radiodiagnostic compounds, radiopharmaceuticals, drugs, toxins, and other agents to proteins, one skilled in the art will be able to determine a suitable method for attaching a given agent to a modified glycoprotein.

In another embodiment of the invention, each modified glycoprotein hormone can have the same or a different agent attached thereto. Any suitable combination of agents can be used selected from the group consisting of radionuclides, drugs, toxins, viruses, cytoprotective compounds, antibodies, sensitizers and biological response modifiers.

C Methods of Detecting an Analyte

In one embodiment, the methods provide for the detection of an analyte that interferes with the binding of a modified glycoprotein hormone receptor in a biological sample, said method comprising (i) contacting the sample, with a modified glycoprotein hormone, said modified glycoprotein hormone having at least one mutation that increases the hormone activity relative to the wild type glycoprotein hormone and (ii) detecting a signal wherein the presence or amount of the signal detected indicates the presence or absence of an analyte that interferes with the binding of a modified glycoprotein hormone to a glycoprotein receptor.

In one embodiment, the method for the detection of an analyte is a competitive binding assay. A competitive binding assay is an assay based on the competition between a labeled and an unlabelled ligand in the reaction with a receptor binding agent (e.g., antibody, receptor, transport protein). IUPAC Compendium of Chemical Terminology, 1997, 2nd edition, “Competitive Protein Binding Assays” Odell and Daughaday, W. H. Lippincott, 1972 and “Principles of Competitive Protein-binding Assays” Odell and Franchimont, P. John Wiley & Sons Inc., 1983, incorporated herein by reference in their entireties. See also, U.S. Pat. No. 6,537,1760, incorporated herein by reference in its entirety.

In certain embodiments, the signal is the presence or amount of the modified glycoprotein hormone bound with the glycoprotein receptor in the sample. In certain embodiments, the method employs the detection of a secondary signal, such as, for example, the detection of the presence or amount of cAMP or a steroid (e.g., progesterone). In certain embodiments, the signal is the presence, absence or amount of inositol triphosphate or other component of the inositol phosphate pathway. In certain embodiments, the signal is the presence or amount of intracellular calcium or the activity of calcium-dependent kinases, or a combination thereof. In certain embodiments, the signal is the presence, amount or activity of protein kinase B (PKB) or serum/glucocorticoid-induced kinase. (SgK).

In certain embodiments, the methods employ the use of whole cells in the biological sample. In certain embodiments, the methods employ only parts of cells, for example, cell membranes.

In certain embodiments, the methods provide for the detection of an analyte, wherein the analyte is an antibody to an extracellular domain of a glycoprotein receptor. For example, circulating extracellular domains of thyroid stimulating hormone receptor have been implicated in the etiology of Graves' disease. See, Fan, et al., Autoimmunity 1993, 15(4): 285-91, Seetharamaiah, et al., Thyroid 19919, 9(9): 879-86, Kikuoka et al., Endocrinology 1998, 139(4): 1891-8, Cho, J. Korean Med. Sci. 2002, 17(3): 293-301 and Cornelia, et al., Biochemistry 2001, 40(33): 9860-9, incorporated herein by reference in their entireties. Such receptor fragments can result in enhanced anti-TSHR antibody titer. Without being bound by any theory, it is believed that the high affinity of the modified glycoprotein hormones, described herein, together with highly specific glycoprotein receptor antibodies could bind with greater specificity and higher affinity to glycoprotein receptor fragments providing an improved method of detecting such receptor fragments. In addition, comparative assays using high-affinity glycoprotein analogs and extracellular domains of glycoprotein receptors may provide a sensitive tool for detecting and measuring anti-extracellular domain antibodies. The detection of such extracellular domain receptor fragments and receptor-specific antibodies could provide early detection of, for example, Graves' disease. In certain embodiments, the methods provide for the monitoring of Graves' disease or to prevent the progression of Graves' disease. In certain embodiments, the detection of such modified glycoprotein hormone-Ab bound to receptor fragments can diagnose, detect or explain idiopathic infertility. See, for example, Kubo, et al., Endocrin. J. 2000, 47(2): 197-201, Mimura, et al, Endocr. J. 2001, 48(2): 255-60 and Kung, et al., J. Clin. Endocrinol. Metab. 2001, 86(8): 3647-53, incorporated herein by reference in their entireties, discussing the association of thyroid antibodies with fertility and pregnancy.

As described supra, without being bound by any theory, it is expected that a specific small sub-group of subjects will benefit the most from the methods of invention. These subjects are those with decreased glycoprotein hormone receptor binding due to mutations that decrease glycoprotein hormone binding and/or glycoprotein hormone receptor expression. High affinity glycoprotein analogs, such as the modified glycoproteins described herein, are expected to overcome, at least in part, limitations of targeted delivery of an agent in such sub-group of subjects.

In certain embodiments, the assay can be performed in solution. In certain embodiments, one or more components of the assay can be immobilized on a solid phase. Plastic surfaces, microparticles, magnetic particles, filters, polymer gel materials and other solid-phase substrates can be used as solid phases. See, for example, U.S. Pat. Nos. 6,664,114; 6,589,798; 6,479,296 and 6,294,342, incorporated herein by reference in their entireties. It is possible to automate the methods of assay provided in the invention.

In the methods of the invention, the manner of incubation (i.e., the method of contacting the biological sample with the modified glycoprotein hormone and subsequent handling prior to detection) are not of import. For example, in some methods of assay, following the contact of a biological sample and a binding competitor, removal of supernate is required. In other methods of assaying, a wash step is often required following the contacting of the biological sample with a solid phase bound binding competitor. The methods of the present invention are not limited to any one manner of incubation.

The biological sample used in the methods of the present invention can be from any animal fluid, including but not limited to, whole blood, serum, plasma, urine, saliva, spinal fluid or fecal matter.

D. Modified Glycoprotein Hormones

The methods of imaging, targeted delivery of an agent and assaying, described supra, employ modified glycoprotein hormones. Certain amino acid residues in the wild type glycoprotein hormone structure can be replaced with other amino acid residues without significantly deleteriously affecting, and in many cases even enhancing, the activity of the glycoprotein hormones. Such modified glycoprotein hormones have been described in U.S. Pat. No. 6,361,992, U.S. application Ser. Nos. 10/057,113 (filed Jan. 25, 2002), 09/813,398 (filed Mar. 20, 2001) and U.S. Provisional Application No. ______ (Attorney Docket No. 56815-5001 PR) (filed Mar. 19, 2004) and PCT Publications 00/17360, 97/42322 and 96/06483, the contents of which are hereby incorporated by reference in their entireties.

In one embodiment, the modified glycoprotein hormones have at least one, at least two, at least three, at least four or at least five defined amino acid residues in the α-subunit substituted with another amino acid residue. In one embodiment, the modified glycoprotein hormones have at least one, at least two, at least three, at least four or at least five defined amino acid residues in the β-subunit substituted with another amino acid residue. In certain embodiments, the modified glycoprotein hormones are modified TSH, modified FSH, modified LH or modified CG.

In certain preferred embodiments, the invention provides imaging, targeting delivery and assay methods using a modified TSH comprising at least one, at least two, at least three, at least four or at least five basic amino acids in the α-subunit at positions selected from the group consisting of positions 11, 13, 14, 16, 17, 20 and 22. In certain preferred embodiments, the invention provides imaging, targeting delivery and assay methods using a modified TSH comprising at least one, at least two, at least three, at least four, at least five, at least six, at least seven or at least eight basic amino acid in each of positions 1, 6, 17, 58, 63, 66, 69 and 81 of the β-subunit. In certain embodiments, the basic amino acids are lysine or arginine.

In certain preferred embodiments, the invention provides imaging, targeting delivery and assay methods using a modified FSH comprising at least one, at least two, at least three, at least four, at least five, at least six, at least seven, at least eight, at least nine, at least ten, at least eleven or at least twelve basic amino acids in the α-subunit at positions 13, 14, 16, 17, 20, 21, 22, 66, 68, 73, 74 and 81. In certain preferred embodiments, the invention provides imaging, targeting delivery and assay methods using a modified FSH comprising at least one, at least two, at least three, at least four, at least five, at least six, at least seven basic amino acids in the β-subunit at positions 2, 4, 14, 63, 64, 67 and 69. In certain embodiments, the basic amino acids are lysine or arginine.

E. Disorders Encompassed by the Methods of the Invention

As described above, the family of glycoprotein hormones derive from the anterior pituitary and exert their effects on glycoprotein hormone receptors in various tissues, particularly the thyroid and organs of the reproductive system. The association of disorders involving alterations in the glycoprotein hormones sharing this pituitary-hypothalamic axis are known, particularly the association of breast cancer with thyroid disorders. See, Mittra, Br. Med. J. 1976, 1: 257-259, Ito and Maruchi, Lancet 1975, 2:1119-1121, Kapdi and Wolfe, JAMA 1976, 236:1124-1127, Rasmusson, et al., J. Cancer Clin. Oncol. 1987, 23:553-556, Mittra and Haysward, Lancet 1974, 1:885-888, Shering, et al., Eur. J. Cancer Prev. 1996, 5:504-506, Maruchi, et al., Mayo Clin. Proc. 1976, 51:263-265, Lemmarie and Baugnet-Mahieu, Eur. J. Cancer Clin. Oncol. 1986, 22:301-307, Moossa, et al., Ann. R. Coll. Surg. 1973, 53:178-188, Kurland and Annegers Lancet 1976, 1:808, Anker, et al., Scand. J. Clin. Lab Invest. 1998, 58:103-107, Smyth, et al., J. Clin. Endocrinol. Metabol. 1996, 81: 937-941, Goldman, Epidemiology Rev. 1990, 12:28-30, McThernan, et al., Cancer Res. 1987, 47:292-294, Ron, et al., Br. J. Cancer 1984, 49:87-90, Gogas, et al., Eur. J. Surg. Oncol. 2001, 27: 626-630, Myhil, et al., Acta Endocrinol. 1966, 51:290-300, Giani et al., J. Endocr. Metab. 1986, 81:990-994, Smyth et al., Clin. Endocr. Metab. 1988, 83:2711-2716, Smyth, J. Endocrinol. Invest. 2000, 23:42-43, Davies, J. Clin. Endocrinol. Metabol. 1994, 79:1232-1238, Dumont and Maenhaut, Baillieres Clin. Endocrinol. Metabol. 1991, 5:727-753, Spitzweg, et al., J. Clin. Endocrinol. Metab. 1998, 83:1746-1751 and Kilbane, et al., J. Endocrinol. 1998, 156:323, incorporated herein by reference in their entireties.

In one embodiment, the methods of the invention provide, inter alia, for the imaging of cells comprising a glycoprotein hormone receptor. In one embodiment, cells comprising a glycoprotein hormone receptor are cells present in disorders such as thyroid cancer, Graves' disease, Hashimoto's disorder, ovarian cancer, cervical cancer, endometrial cancer, lung cancer, teratomas, breast cancer, testicular cancer or pituitary tumor. The methods provide for the imaging of disorders associated with thyroid disease, including autoimmune disorders, and cancers affecting the pituitary-hypothalamic axis or gonadal tissues.

The invention also provides methods, inter alia, for delivering an agent to a cell expressing a glycoprotein receptor to a subject in need thereof. In one embodiment, cells expressing a glycoprotein receptor are cells present in disorders such as thyroid cancer, Graves' disease, Hashimoto's disorder, ovarian cancer, cervical cancer, endometrial cancer, lung cancer, teratomas, breast cancer, testicular cancer or pituitary tumor. The methods provide for the delivery of an agent to a subject suffering from or suspected of suffering from disorders associated with thyroid disease, including autoimmune disorders, and cancers affecting the pituitary-hypothalamic axis or gonadal tissues.

The invention further provides methods, inter alia, for detecting an analyte that interferes with the binding of a modified glycoprotein hormone to a glycoprotein receptor. In one embodiments, the presence or absence of an analyte that interferes with the binding of a modified glycoprotein hormone to a glycoprotein receptor can be associated with disorders such as thyroid cancer, Graves' disease, Hashimoto's disorder, ovarian cancer, cervical cancer, endometrial cancer, lung cancer, teratomas, breast cancer, testicular cancer, pituitary tumor, ovulatory dysfunction, luteal phase defect, unexplained infertility, male factor infertility, time-limited conception or spontaneous abortion. The methods provide for the detection of an analyte in a biological sample from a subject suffering from or suspected of suffering from disorders associated with thyroid disease, including autoimmune disorders, and cancers affecting the pituitary-hypothalamic axis or gonadal tissues are within the scope of the present invention. In addition, the methods provide for the detection of an analyte in a biological sample from a subject suffering from or suspected of suffering from disorders associated with infertility or difficulties in conceiving or maintaining pregnancy.

F. Administration, composition and Dosing

The modified glycoprotein hormones or compositions thereof can be administered by any suitable route that ensures bioavailability in the circulation. This can best be achieved by parenteral routes of administration, including intravenous (IV), intramuscular (IM), intradermal, subcutaneous (SC) and intraperitoneal (IP) injections. However, other routes of administration can be used. For example, absorption through the gastrointestinal tract can be accomplished by oral routes of administration (including but not limited to ingestion, buccal and sublingual routes) provided appropriate formulations (e.g., enteric coatings) are used to avoid or minimize degradation of the active ingredient, e.g., in the harsh environments of the oral mucosa, stomach and/or small intestine. In some instances, such as when imaging the gastrointestinal tract, absorption is not required. In these instances, the modified glycoprotein hormones are not absorbed from the gastrointestinal tract. Alternatively, administration via mucosal tissue such as vaginal and rectal modes of administration can be utilized to avoid or minimize degradation in the gastrointestinal tract. In one alternative, the modified glycoprotein hormones or compositions thereof can be administered transcutaneously (e.g., transdermally), or by inhalation. It will be appreciated that the preferred route may vary with the condition, age, overall health of the subject, the suspected disorder and the type of imaging to be performed.

The actual amount of the modified glycoprotein hormones or compositions thereof to be administered will vary with the route of administration, and the purpose for the administration (e.g., imaging or targeted delivery of an agent). The amount to be administered can be determined by one of skill in the art (e.g., a radiologist or oncologist) taking into consideration the age, overall health and medical condition of the subject. See, Remingtons Pharmaceutical Sciences, Mack Publishing Co. (A. R. Gennaro ed. 1985).

The dose of radionuclides can be determined by one of skill in the art. Radionuclide dosing is expressed in terms of radioactivity emitted. The radionuclides can be administered to a subject as a dose of about 0.01 to about 1,000 mCi. In a preferred embodiment, the dose of a radionuclide is about 0.1 to about 500 mCi. In a more preferred embodiment, the dose of a radionuclide is about 1 to about 100 mCi. In a more preferred embodiment, the dose of a radionuclide is about 5 to about 80 mCi. In a most preferred embodiment, the dose of a radionuclide is about 50 mCi. See, Tuttle, et al., Thyroid 1995, 5(4):243-7, Degrossi, et al., Eur. J. Clin. Pharmacol. 1995, 48(6):489-94 and DiRusso and Kearn, Surgery 1994, 116(6):1024-30, incorporated herein by reference.

### 6. EXAMPLES

The following is a prophetic example of how therapeutic agents could be delivered to a thyroid cancer cell, particularly TSH receptor (TSHR)-mediated delivery to a thyroid cancer cell. FIG. 1 provides a schematic depicting a thyroid cancer cell with thyroid stimulating hormone receptors (TSHR) on its surface. The modified glycoprotein hormones, identified as a high affinity TSH analog and depicted as two-linked gray ellipses representing subunits, is coupled to a retrovirus that is coupled to or expresses sodium iodide symporter (NIS), TSHR, toxins or p53. In this scenario, the high affinity of the modified TSH provides specific binding to the TSHR. The coupled agents are thus delivered to the vicinity of the thyroid cancer cell to exert their desired effect.

In one scenario cancer cell differentiation could be restored using high affinity interaction between a TSH analog and the largely depleted pool of TSH receptors. In one scenario, it is hypothesized that delivery of genetic material can be facilitated by the high affinity interaction between the modified glycoprotein hormones described herein and the glycoprotein hormone receptors. In such a scenario, genetic material can be coupled to a modified glycoprotein hormone for targeted delivery to a cancerous cell. The uptake of this genetic material would increase the number of receptors and restore cell differentiation. It is also hypothesized that delivery of a modified glycoprotein hormone to a cancerous cell, for example, delivery of modified TSH to a thyroid cancer cell, will increase the number of TSH receptors expressed on the thyroid cancer cell. Such increased expression of TSH receptors would stimulate or restore cell differentiation or facilitate killing of the thyroid cancer cell by providing an increased number of targets (e.g., TSH receptors).

The disclosures of all publications referenced throughout this application are hereby incorporated by referenced in their entireties. The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description. Such modifications are intended to fall within the scope of the appended claims.

What is claimed is:

1. A method of imaging cells comprising a glycoprotein hormone receptor, said method comprising administering to a subject a modified glycoprotein hormone, said modified glycoprotein hormone having at least one mutation that increases the hormone activity relative to the wild type glycoprotein hormone and detecting said modified glycoprotein hormone.

2. The method of claim 1 wherein the modified glycoprotein hormone is a modified thyroid stimulating hormone (TSH).

3. The method of claim 1 wherein the modified glycoprotein hormone is a modified follicle-stimulating hormone (FSH).

4. The method of claim 1 wherein the modified glycoprotein hormone is a lutenizing hormone (LH).

5. The method of claim 1 wherein the modified glycoprotein hormone is chorionic gonadotropin (CG).

6. The method of claim 2 wherein the modified TSH differs from the wild type TSH in that the modified TSH α-subunit comprises at least one basic amino acid at positions selected from the group consisting of 11, 13, 14, 16, 17, 20 and 22.

7. The method of claim 6 wherein the modified TSH comprises at least one basic amino acid at position 1, 6, 17, 58, 63, 66, 69 and 81 of the β-subunit.

8. The method of claim 6 wherein the modified TSH comprises at least three basic amino acids at positions 11, 13, 14, 16, 17, 20 or 22 of the α-subunit.

9. The method of claim 6, 7 or 8 wherein the basic amino acids are lysine or arginine.

10. The method of claim 1 wherein the cells comprising a glycoprotein hormone receptor are cancerous cells or cells indicative of an autoimmune disorder.

11. The method of claim 1 wherein detecting increased levels of said modified glycoprotein hormone in said subject indicates the presence of cancerous cells or an autoimmune disorder.

12. The method of claim 11 wherein the cancerous cells are thyroid carcinoma cells.

13. The method of claim 11 wherein the cancerous cells are selected from the group consisting of ovarian cancer, uterine cancer, cervical cancer, endometrial cancer, lung cancer, teratomas, breast cancer, testicular cancer or pituitary tumor.

14. The method of claim 1 wherein the autoimmune disorder is Graves' disease or Hashimoto's disorder.

15. The method of claim 1 wherein said modified glycoprotein hormone is labeled.

16. The method of claim 15 wherein the label is a radiopaque label, radioisotope label, fluorescence label or paramagnetic label.

17. The method of claim 16 wherein the radiopaque label is an ionic or nonionic agent.

18. The method of claim 17 wherein the ionic agent is selected from the group consisting of diaztrizoate meglumine 30%, diaztrizoate meglumine 60%, diaztrizoate meglumine 66%, diaztrizoate sodium 10%, diaztrizoate sodium 50%, iothalamate meglumine 30%, iothalamate meglumine 43%, iothalamate meglumine 60%, ioxaglate meglumine 39.3%, iothalamate sodium 19.6% or combinations thereof.

19. The method of claim 17 wherein the nonionic agent is selected from the group consisting of gadodiamide, gadoteridol, gadoversetamide, iodixanol 270, iodixanol 320, iohexyl 140, iohexyl 180, iohexyl 240, iohexyl 300, iohexyl 350, iopamidol 410%, iopamidol 51%, iopamidol 61%, iopamidol 76%, iopromide 150, iopromide 240, iopromide 300, iopromide 370, ioversol 34%, ioversol 51%, ioversol 64%, ioversol 68%, ioversol 74% or combinations thereof.

20. The method of claim 16 wherein the radioisotope label is I¹³¹ or Tc^{99m}.

21. The method of claim 16 wherein the paramagnetic label is gadodiamide, gadoteridol, gadoversetamide, ferumoxides, gadopentetate dimeglumine, mangafodipir tridosium, or combinations thereof.

22. The method of claim 1 further comprising administration of protirelin, thyrotropin alpha, gonadorelin or combinations thereof.

23. The method of claim 15 wherein the labeled modified glycoprotein hormone is detected by a method selected from group consisting of magnetic resonance imaging, computed tomography imaging, nuclear medicine imaging, X-ray, mammography, radionuclide imaging or combinations thereof.

24. The method of claim 15 wherein detecting an amount of said labeled modified glycoprotein hormone in said subject indicates the presence of cancerous cells or an autoimmune disorder.

25. The method of claim 24 wherein the cancer is thyroid cancer.

26. The method of claim 24 wherein the cancer is selected from the group consisting of ovarian cancer, uterine cancer, cervical cancer, endometrial cancer, lung cancer, teratomas, breast cancer, testicular cancer or pituitary tumor.

27. The method of claim 24 wherein the autoimmune disorder is Graves' disease or Hashimoto's disorder.

28. A method of delivering an agent to a cell expressing a glycoprotein receptor to a subject in need thereof, said method comprising administering to said subject an agent coupled to a modified glycoprotein hormone having at least one mutation that increases the hormone activity relative to the wild type glycoprotein hormone.

29. The method of claim 28 wherein the modified glycoprotein hormone is a modified TSH.

30. The method of claim 28 wherein the modified glycoprotein hormone is a modified FSH.

31. The method of claim 28 wherein the modified glycoprotein hormone is a modified LH.

32. The method of claim 28 wherein the modified glycoprotein hormone is modified CG.

33. The method of claim 29, wherein the modified TSH differs from the wild type TSH in that the modified TSH α-subunit comprises at least one basic amino acid at positions selected from the group consisting of 11, 13, 14, 16, 17, 20 and 22.

34. The method of claim 29 wherein the modified TSH comprises at least one basic amino acid at position 1, 6, 17, 58, 63, 66, 69 and 81 of the β-subunit.

35. The method of claim 29 wherein the modified TSH comprises at least three basic amino acids at positions 11, 13, 14, 16, 17, 20 or 22 of the α-subunit.

36. The method of claim 33, 34 or 35 wherein the basic amino acids are lysine or arginine.

37. The method of claim 28 wherein said agent is selected from the group consisting of cytoprotective compounds, antibodies, drugs, sensitizers, biological response modifiers, radionuclides, toxins, viruses or combinations thereof.

38. The method of claim 37 wherein the agent is a drug selected from the group consisting of natural or synthetic estrogens, estrogen receptor modulators, progestins, androgens, ovulation stimulants, gonadotropin-releasing hormones, androgen inhibitors, bisphosphonates, glucocorticoids, thyroid hormones, antithyroid agents, alkylating agents, antimetabolites, antimitotic agents, epipodophyllotoxins, antineoplastic antibiotics, antineoplastic hormones, platinum coordination complex agents, anthracenediones, substituted ureas, methylhydrazine derivatives, DNA topoisomerase inhibitors, retinoids, or combinations thereof.

39. The method of claim 38 wherein the drug is selected from the group consisting of clomiphene, finasteride, propylthiouracil, methimazole, bleomycin, vincristine, vinblastine, cisplatin, mitomycin, ifosfamide, cyclophosphamide, doxorubicin, paclitaxel, fluorouracil, carboplatin, epirubicin, altretamine, vinorelbine, mitoxantrone, bromocriptine prednisone, porfimer, mitotane or combinations thereof.

40. The method of claim 38 wherein the sensitizer is selected from the group consisting of metronidazole, misonidazole, verapamil, diltiazern or combinations thereof.

41. The method of claim 37 wherein the agent is a biological response modifier selected from the group consisting of interferon-α, interferon-β, interferon-γ, tumor necrosis factor, lymphotoxin, interleukin-1, interleukin-2, interleukin-3, interleukin-4, interleukin-5, interleukin-6, p53 or combinations thereof.

42. The method of claim 37 wherein the agent is a monoclonal antibody, polyclonal antibody or combination thereof.

43. The method of claim 37 wherein the agent is a cell signal transduction pathway modifier.

44. The method of claim 43 wherein the agent is selected from the group consisting of forskolin, staurosporine, phorbol esters, non-steroidal antiinflammatory drugs, steroids, or combinations thereof.

45. The method of claim 37 wherein the agent is a cytoprotective compound.

46. The method of claim 43 wherein the cytoprotective compound is mesna or leucovorin.

47. The method of claim 37 wherein the radionuclide is selected from the group consisting of ¹³¹I, ¹³²I, ³²P, ¹⁸⁶Re, ¹⁸⁸Re, ²⁰³Pb, ²¹²Pb, ²¹²Bi, ¹⁰⁹Pd, ⁶⁴Cu, ⁶⁷Cu, ²¹¹At, ⁹⁷Ru, ¹⁰⁵Rh, ¹⁹⁸Au and ¹⁹⁹Au.

48. The method of claim 37 wherein the toxin is ricin, abrin, diphtheria toxin, Pseudomonas exotoxin A, ribosomal inactivating proteins, and mycotoxins.

49. The method of claim 37 wherein the viruses are selected from the group consisting of adenovirus, retrovirus or combinations or fragments thereof.

50. The method of claim 28 wherein the subject has or is suspected of having a disorder selected from the group consisting of thyroid cancer, Graves' disease, Hashimoto's disorder, ovarian cancer, uterine cancer, cervical cancer, endometrial cancer, lung cancer, teratomas, breast cancer, testicular cancer or pituitary tumor.

51. A method for the detection of an analyte that interferes with the binding of a modified glycoprotein hormone to a glycoprotein receptor in a biological sample, said method comprising (i) contacting the sample, with a modified glycoprotein hormone, said modified glycoprotein hormone having at least one mutation that increases the hormone activity relative to the wild type glycoprotein hormone and (ii) detecting a signal wherein the presence or amount of the signal detected indicates the presence or absence of an analyte that interferes with the binding of a modified glycoprotein hormone to a glycoprotein receptor.

52. The method of claim 51 wherein the signal is the presence or amount of the modified glycoprotein hormone bound with the glycoprotein receptor in the biological sample.

53. The method of claim 51 wherein the signal is the presence or amount of cAMP in the biological sample.

54. The method of claim 51 wherein the signal is the presence or amount of steroids in the biological sample.

55. The method of claim 54 wherein the signal is the presence or amount of progesterone in the biological sample.

56. The method of claim 51 wherein the signal is the presence or amount of inositol trisphosphate or other component of inositol phosphate pathway.

57. The method of claim 51 wherein the signal is the presence or amount of intracellular calcium, activity of calcium-dependent kinases or a combination thereof.

58. The method of claim 51 wherein the signal is the presence or activity of protein kinase B (PKB) or serum/glucocorticoid-induced kinase (Sgk).

59. The method of claim 51 wherein the modified glycoprotein hormone is a modified TSH.

60. The method of claim 51 wherein the modified glycoprotein hormone is a modified FSH.

61. The method of claim 51 wherein the modified glycoprotein hormone is a modified LH.

62. The method of claim 51 wherein the modified glycoprotein hormone is modified CG.

63. The method of claim 59 wherein the modified TSH comprises at least one basic amino acid at a position selected from the group consisting of II, 13, 14, 16, 17, 20 and 22 of the α-subunit.

64. The method of claim 59 wherein the modified TSH comprises at least one basic amino acid at a position selected from the group consisting of 1, 6, 17, 58, 63, 66, 69 and 81 of the β-subunit.

65. The method of claim 60 wherein the modified FSH comprises at least one basic amino acid at a position selected from the group consisting of 13, 14, 16, 17, 20, 21, 22, 66, 68, 73, 74 and 81 of the α-subunit.

66. The method of claim 60 wherein the modified FSH comprises at least one basic amino acid at a position selected from the group consisting of 2, 4, 14, 63, 64, 67 and 69 of the β-subunit.

67. The method of claim 63, 64, 65 or 66 wherein the basic amino acids are lysine or arginine.

68. The method of claim 51 wherein the analyte is an antibody to a glycoprotein receptor.

69. The method of claim 51 wherein the analyte is an antibody to a glycoprotein hormone receptor extracellular domain.

70. The method of claim 51 wherein the analyte is wild type glycoprotein hormone.

71. The method of claim 51 wherein the glycoprotein receptor is selected from the group consisting of receptors for TSH, FSH, LH, CG or combinations thereof.

72. The method of claim 51 wherein said modified glycoprotein hormone is labeled.

73. The method of claim 51 wherein the biological sample comprises whole cells.

74. The method of claim 51 wherein the biological sample comprises cell membranes.

75. The method of claim 51 wherein the detection of the signal indicates that the subject from whom the biological sample was acquired is suffering from a disorder selected from the group consisting of thyroid cancer, Graves' disease, Hashimoto's disorder, ovarian cancer, uterine cancer, endometrial cancer, lung cancer, teratomas, breast cancer, testicular cancer, pituitary tumor, ovulatory dysfunction, luteal phase defect, unexplained infertility, male factor infertility, time-limited conception or spontaneous abortion.
